(19) United States
(12) Patent Application Publication
(10) Pub. No.: US 2025/0114058 A1
(43) Pub. Date: Apr. 10, 2025

(54) RADIOGRAPHY DEVICE AND RADIOGRAPHY SYSTEM

(71) Applicant: CANON KABUSHIKI KAISHA, Tokyo (JP)

(72) Inventor: YOHEI SAITO, Kanagawa (JP)

(21) Appl. No.: 18/903,896

(22) Filed: Oct. 1, 2024

(30) Foreign Application Priority Data
Oct. 6, 2023 (JP) 2023-174657

(51) Int. Cl. A61B 6/00; A61B 6/10

(52) U.S. Cl. CPC A61B 6/4429; A61B 6/107; A61B 6/52; A61B 6/563; A61B 6/4405; A61B 6/4411

(57) ABSTRACT

A radiography device includes a sensor panel that generates image information from incident radiation, an antenna that wirelessly transmits the image information generated by the sensor panel, and a housing that houses the sensor panel and the antenna, wherein the housing has a recessed portion recessed toward an inside, the recessed portion having a transmission portion configured to transmit an electromagnetic wave, and the antenna is disposed to face the transmission portion.

### BACKGROUND

### Field

The present disclosure relates to a radiography device and a radiography system.

### Description of the Related Art

Conventionally, radiography devices that send radiation, such as X-rays, to a test object and visualize the distribution of the intensity of transmitted radiation are widely used in medical diagnosis.

In recent years, such a radiography device is sometimes used in combination with a portable radiation sensor in which a sensor portion converting radiation into image signals is driven by a battery and the image signals are transmitted wirelessly. The limit value of a specific absorption rate (SAR) is often set legally for such devices that perform wireless communication to regulate impacts on the human body. It should be noted that the SAR refers to the amount of energy absorbed per unit time by a unit mass of tissue when the human body is exposed to electromagnetic waves.

One general method for reducing the SAR value is, for example, to use electromagnetic wave absorption materials. Use of this measure reduces the SAR value but causes problems, such as degradation of communication performance and need for additional members.

The structure described in Japanese Patent Laid-Open No. 2022-187050 illustrates a method for reducing the SAR value without need for additional constituent members. However, this method can reduce the SAR value on the incident surface of radiation but has problems in that reduction in the SAR value is insufficient.

### SUMMARY

The present disclosure provides a radiography device and a radiography system that enable miniaturization and weight reduction of the device without the need for additional constituent members as SAR measures and can sufficiently reduce the SAR value by reducing adverse effects of communication electromagnetic waves on a test subject and the like.

A radiography device according to an aspect of the present disclosure includes a sensor panel that generates image information from incident radiation, an antenna that wirelessly transmits the image information generated by the sensor panel, and a housing that houses the sensor panel and the antenna, in which the housing has a recessed portion recessed toward an inside, the recessed portion having a transmission portion that transmits an electromagnetic wave, and the antenna is disposed to face the transmission portion.

Further features of the present disclosure will become apparent from the following description of exemplary embodiments with reference to the attached drawings.

### BRIEF DESCRIPTION OF THE DRAWINGS

FIGS. 1A, 1B, and 1C are schematic diagrams illustrating a radiography device (X-ray sensor) according to a first embodiment.

FIGS. 2A, 2B, and 2C cross-sectional views illustrating radiation images of electromagnetic waves radiated from an antenna indicated by arrows in the X-ray sensor according to the first embodiment.

FIGS. 3A, 3B, and 3C are schematic diagrams illustrating a radiography device (X-ray sensor) according to a second embodiment.

FIGS. 4A, 4B, and 4C are schematic diagrams illustrating enlarged view of the vicinity of an antenna window in the second embodiment.

FIGS. 5A and 5B are schematic diagrams illustrating X-ray photography of a knee portion of a test subject by using the X-ray sensor according to the second embodiment.

FIGS. 6A and 6B are schematic diagrams illustrating a case in which X-ray photography of a chest portion is performed by using the X-ray sensor.

FIG. 7 is a diagram illustrating the back surface of the X-ray sensor according to the second embodiment.

FIGS. 8A, 8B, and 8C are schematic diagrams illustrating an X-ray photography device (X-ray sensor) according to a third embodiment.

FIGS. 9A, 9B, and 9C are schematic diagrams illustrating enlarged view of the vicinity of an antenna window in the third embodiment.

FIGS. 10A and 10B are schematic diagrams illustrating X-ray photography of a foot portion of the test subject by using the X-ray sensor according to the third embodiment.

FIGS. 11A, 11B, and 11C are schematic diagrams illustrating an X-ray photography device (X-ray sensor) according to a fourth embodiment.

FIG. 12 is a schematic diagram illustrating a radiography system.

### DESCRIPTION OF THE EMBODIMENTS

Individual embodiments to which the present disclosure is applicable will be described below with reference to the drawings. It should be noted that constituent members common to a plurality of drawings are denoted by common reference numerals in the following description. Accordingly, constituent members common to a plurality of drawings will be described and constituent members denoted by common reference numerals will not be described as appropriate. Radiation in individual embodiments nay include beams of particles (including photons) released by radioactive decay, such as alpha, beta, and gamma rays as well as beams with equal or greater energy, such as X-rays, corpuscular rays, and cosmic rays, and the like. In the embodiments below, X-rays are used as radiation.

### First Embodiment

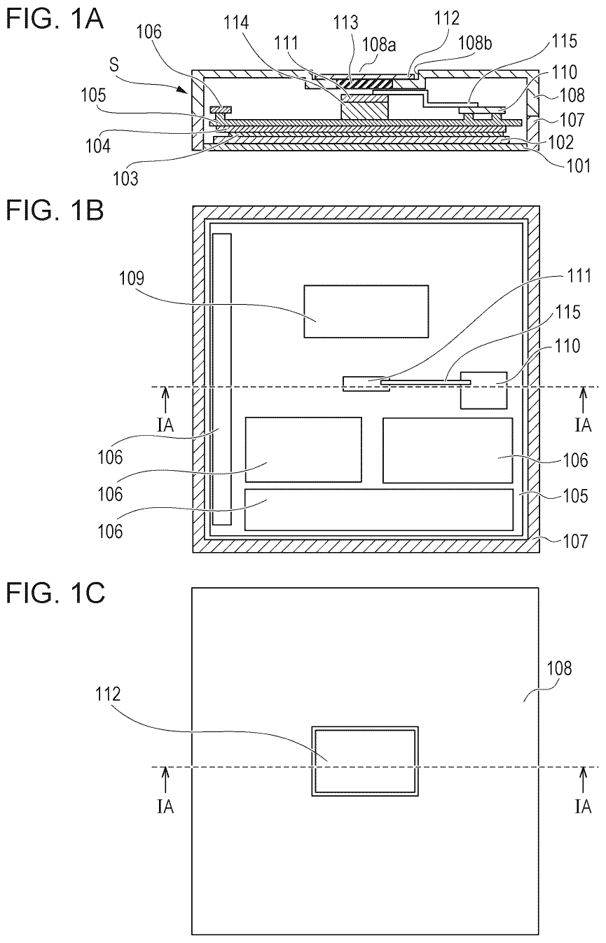

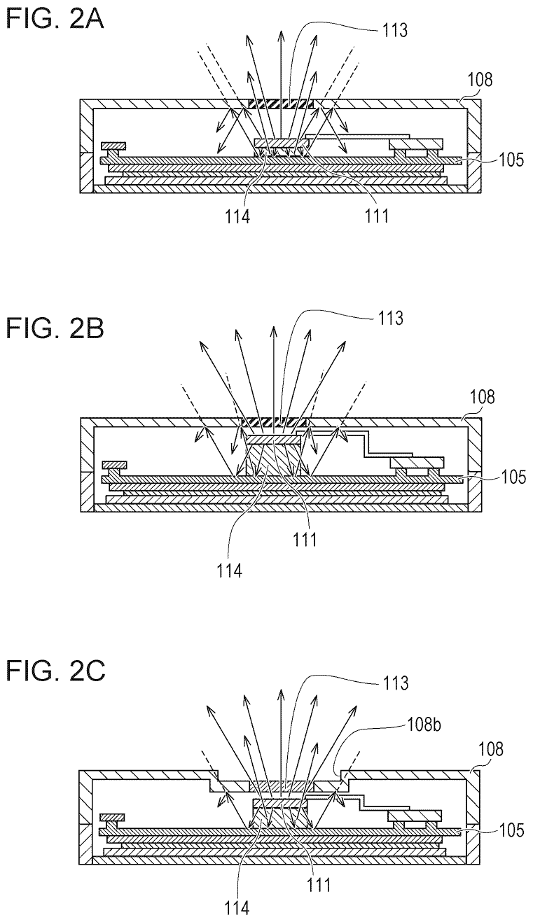

A first embodiment will be described with reference to FIGS. 1 and 2.

FIGS. 1A, 1B, and 1C are schematic diagrams illustrating a radiography device (X-ray sensor) according to the present embodiment. FIG. 1A is a cross-sectional view taken along dotted line IA-IA in FIGS. 1B and 1C, FIG. 1B is a plan view illustrating an internal structure as viewed from the back surface of an X-ray incident surface, and FIG. 1C is a plan as view from the back surface.

As illustrated in FIG. 1A, the X-ray sensor S includes, as constituent members, an incident surface plate 101 that serves as an incident surface, an impact absorbing member 102, a fluorescence substance 103, a sensor panel 104, and a support base 105 in this order from the incident surface on which an X-ray having been transmitted through the test object is incident.

The incident surface plate 101 is formed of a material, such as carbon fiber reinforced plastics (CFRP) with good X-ray transmittance and high rigidity. The impact absorbing member 102 is formed of, for example, a foam material or the like through which an impact from the incident surface is less likely to be transmitted to be the inside.

The fluorescence substance 103 is a member made of a material that converts an incident X-ray into visible light, such as CsI or GOS and is attached to the impact absorbing member 102. The sensor panel 104 includes, for example, a sensor board on which many photoelectric-conversion elements are disposed and generates an electrical signal that is image information used to form a two-dimensional image by detecting light emitted by the fluorescence substance 103. The support base 105 is a plate member that is adhered to the sensor panel 104 with an adhesive or the like (not illustrated). The support base 105 is a support plate designed to prevent the sensor panel 104 or the like from being affected by electromagnetic waves radiated from an antenna 111, which will be described later. The support base 105 is formed to include a material that shields electromagnetic waves, such as magnesium alloy, aluminum alloy, a steel material such as stainless steel, and a high-rigidity material such as CFRP.

The X-ray sensor S further includes various electrical boards 106, a wireless module 110, an antenna 111, and a battery 109.

The various electrical boards 106 are provided on the support base 105 as illustrated in FIGS. 1A and 1B and drive the sensor panel 104, visualize electrical signals that form a 2D image, and perform conversion to 2D image information or the like. The various electrical boards 106 are electrically connected to each other by flexible flat cables (FFCs) and the like, which are not illustrated. The wireless module 110 converts image information and various types of information into signals for wireless communication. The antenna 111 communicates with the outside of the X-ray sensor S by releasing, as electromagnetic waves, signals for wireless transmission that have been converted by the wireless module 110. The wireless module 110 and the antenna 111 are electrically connected to each other by a conductive line 115. The battery 109 supplies power to the various electrical boards 106 and sensor panels 104 and is disposed in a detachable manner.

The various constituent members described above are covered with and housed in the housing including a front housing portion 107 and a back housing portion 108. The front housing portion 107 and the back housing portion 108 are connected to each other on the side surfaces of the housing.

The front housing portion 107 is adhered to the incident surface plate 101 by an adhesive, which is not illustrated. The back housing portion 108 is fastened to the front housing portion 107 in a detachable manner by screws or the like, which are not illustrated. Portable X-ray sensors need to have strength to withstand an unintended impact, such as a fall. The front housing portion 107 and the back housing portion 108, which greatly contribute to strength, are often made of a conductive material with excellent strength, such as a metal or CFRP.

In the present embodiment, recessed portions are formed in predetermined portions on the housing surface. Specifically, as illustrated in FIGS. 1A and 1C, in a predetermined portion of the back housing portion 108, that is, in a substantially center portion here, a recessed portion 108b that is recessed toward the inside (toward the incident surface plate 101) and has a rectangular shape in plan view is formed. An opening 108a is formed in the bottom surface that is a bottom portion of the recessed portion 108b, and an antenna window 113 that is a transmission portion through which electromagnetic waves can be transmitted is provided to block the opening 108a. The antenna window 113 is a window portion made of a material mainly including a material through which electromagnetic waves can be transmitted, that is, for example, an insulating material, such as a resin. Since the antenna window 113 (and a display label 112 below) spatially blocks the opening 108a while allowing electromagnetic waves to be transmitted therethrough, electromagnetic waves from the antenna 111 are released to the outside of the X-ray sensor S through the antenna window 113 and the label 112.

The antenna 111 is positionally aligned to face the antenna window 113 and is disposed at substantially the same position as the antenna window 113 in plan view. An antenna base 114 that is a placement portion on which the antenna 111 is placed is disposed on the support base 105, and the antenna 111 is placed on and fixed to the antenna base 114. The antenna 111 is disposed close to the antenna window 113 provided in the bottom surface of the recessed portion 108b by being disposed to face the antenna window 113, but the antenna 111 is disposed closer to the antenna window 113 by the height of the antenna base 114 by the antenna base 114 being provided. Accordingly, by the antenna base 114 with an appropriate height being provided, the antenna 111 can be brought closer to a desired position from the antenna window 113.

The display label 112 is adhered to the recessed portion 108b of the back housing portion 108 with an adhesive or the like that is not illustrated. The display label 112 is made of a material through which electromagnetic waves can be transmitted, such as a resin sheet or paper and descriptions based on various national regulatory requirements, such as the name, the model, the power rating, and the like of the device are printed. The display label 112 has substantially the same size as the recessed portion 108b and is fitted to the recessed portion 108b. When a component like a sheet material is adhered to the outermost surface of the housing of the device with an adhesive, if the user or the like unintentionally comes into contact with an edge portion of the sheet material, the sheet material relatively easily peels off. In the present embodiment, since the display label 112 is fitted and pasted to the recessed portion 108b of the back housing portion 108, the user is less likely to unintentionally come into contact with an end face of the display label 112 and the risk of detachment of the display label 112 is reduced.

Here, the state of electromagnetic waves radiated from the antenna in the X-ray sensor will be described with reference to FIG. 2. FIG. 2 is a cross-sectional view illustrating the radiation image of electromagnetic waves radiated from the antenna in the X-ray sensor by using arrows. FIG. 2A illustrates comparative example 1 in which a typical X-ray sensor is used, FIG. 2B illustrates comparative example 2 in which another typical X-ray sensor, and FIG. 2C illustrates comparative example 3 in which the X-ray sensor according to the present embodiment is used.

As illustrated in FIG. 2A, in comparative example 1, the recessed portion 108b as in the present embodiment is not formed in the back housing portion 108, and the surface of the back housing portion 108 is flat. The thickness of the antenna base 114 is small, and the separation distance between the antenna 111 and the antenna window 113 is relatively large. Electromagnetic waves released from antenna tend to spread in all directions. Accordingly, when the separation distance between the antenna 111 and the antenna window 113 is large, even electromagnetic waves radiated in the direction of the back housing portion 108 of the electromagnetic waves released from the antenna 111 more likely to deviate from the antenna window 113. Such electromagnetic waves are reflected and scattered by the back housing portion 108, which is a conductor, and cannot be released to the outside of the housing.

As illustrated in FIG. 2B, in comparative example 2, the surface of the back housing portion 108 is flat as in comparative example 1, but the thickness of the antenna base 114 is larger than that of comparative example 1, and the separation distance between the antenna 111 and the antenna window 113 is relatively small. When the antenna 111 is disposed closer to the antenna window 113 as described above, the electromagnetic waves radiated in the direction of the back housing portion 108 are efficiently released to the outside of the housing through the antenna window 113. However, when the antenna 111 is disposed closer to the antenna window 113, the antenna 111 is also closer to a user or a test subject, and accordingly, the user or the test subject is possibly adversely affected by the electromagnetic waves.

As illustrated in FIG. 2C, in the present embodiment, the antenna window 113 is provided in the bottom surface of the recessed portion 108b formed in the back housing portion 108, and the antenna 111 is disposed closer to the antenna window 113. In this case, since the antenna 111 is disposed closer to the antenna window 113, the electromagnetic waves radiated in the direction of the back housing portion 108 are efficiently released to the outside of the housing through the antenna window 113. In addition, the distance between the support base 105 and the antenna window 113 is smaller than the distances in comparative examples 1 and 2 because the recessed portion 108b is formed. As a result, the electromagnetic waves reflected by the support base 105 after being radiated in the direction of the incident surface of the X-ray are also efficiently released through the antenna window 113 to the outside of the housing, and the radiation efficiency of the electromagnetic waves is improved. Furthermore, in the present embodiment, since the recessed portion 108b is formed, even when the antenna 111 is disposed closer to the antenna window 113, the antenna 111 is not closer to the user or the test subject and the distance therebetween is maintained, and accordingly, the adverse effects of the electromagnetic waves on the user or the test subject can be reduced.

As described above, according to the present embodiment, it is possible to sufficiently reduce the SAR value by reducing the risk of adverse effects on the human body caused by the electromagnetic waves released from the antenna 111 without adding any special constituent members. Furthermore, electromagnetic waves can be efficiently released from the X-ray sensor S to the outside, and a good communication environment can be obtained.

### Second Embodiment

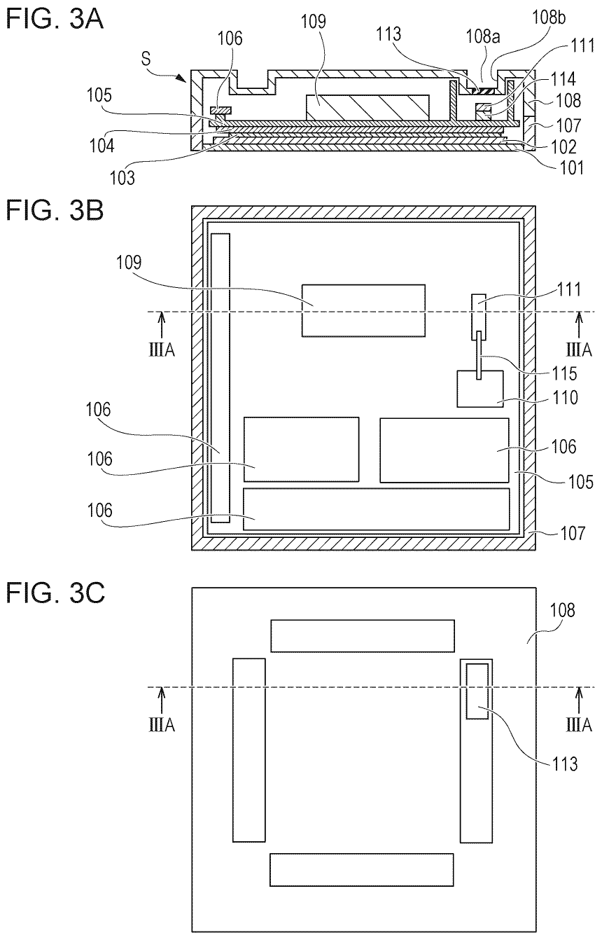

A second embodiment will be described with reference to FIGS. 3 to 6. It should be noted that components identical to those of the first embodiment will not be described.

FIGS. 3A, 3B, and 3C are schematic diagrams illustrating a radiography device (X-ray sensor) according to the present embodiment. FIG. 3A is a cross-sectional view taken along dotted line IIIA-IIIA in FIGS. 3B and 3C, FIG. 3B is a plan view illustrating an internal structure as viewed from the back surface of the X-ray incident surface, and FIG. 3C is a plan view as viewed from the back surface.

In the present embodiment, an X-ray radiography device (X-ray sensor) is disclosed as in the first embodiment, but the present embodiment differs from the first embodiment in the position of the antenna 111, the position of the antenna window 113, and the position of the recessed portion 108b of the back housing portion 108, and the shape of the support base 105.

In the present embodiment, as illustrated in FIG. 3C, four rectangular recessed portions 108b extending along four sides that constitute the outer edge of the back housing portion 108 are formed in the main surface of the back housing portion 108. In the present embodiment, the opening 108a is present in the bottom surface of one of the four recessed portions 108b extending along the right side of the back housing portion 108 in the state illustrated in FIG. 3C, and the antenna window 113 is disposed to block this opening 108a. Here, as in the first embodiment, the antenna 111 is disposed to face the antenna window 113 provided in the bottom surface of the recessed portion 108b, that is, disposed at substantially the same position as the antenna window 113 in plan view. As a result, the antenna 111 is disposed closer to the antenna window 113, but the antenna 111 is closer to the antenna window 113 by the height of the antenna base 114. Accordingly, by providing the antenna base 114 with an appropriate height, the antenna 111 can be brought closer to a desired position toward the antenna window 113 by the antenna base 114 being provided.

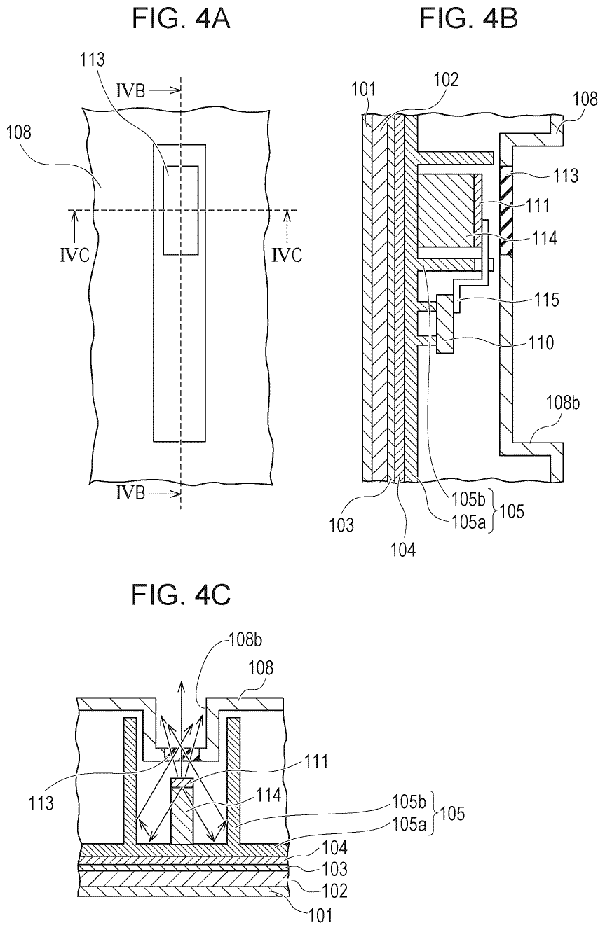

An aspect of the support base 105 in the present embodiment will be described with reference to FIG. 4. FIGS. 4A, 4B, and 4C are schematic diagrams illustrating enlarged view of the vicinity of the antenna window in the present embodiment. FIG. 4A is a plan view, FIG. 4B is a cross-sectional view taken along dotted line IVB-IVB in FIG. 4A, and FIG. 4C is a cross-sectional view taken along dotted line IVC-IVC in FIG. 4A. In FIG. 4C, a radiation image of electromagnetic waves is indicated by arrows.

The support base 105 includes, as a main constituent member, a plate portion 105a made of a material that shields electromagnetic waves, such as magnesium alloy, aluminum alloy, a steel material such as stainless steel, and CFRP, so as to prevent the sensor panel 104 and the like from being affected by electromagnetic waves radiated from the antenna 111.

In the present embodiment, the support base 105 includes the plate portion 105a as well as wall portions 105b formed integrally with the plate portion 105a. The wall portions 105b project from the plate portion 105a toward the back housing portion 108 and is shaped to surround the antenna base 114 and antenna 111.

As illustrated in FIGS. 4A and 4B, portions of the wall portions 105b along the lateral direction of the antenna 111 extend to the vicinity of the back surface of the bottom surface of the recessed portion 108b below the bottom surface and cover both side surfaces in the lateral direction of the antenna 111. Here, a portion of the wall portion 105b has an opening through which a conductive line 115 connecting the antenna 111 and the wireless module 110 to each other passes.

As illustrated in FIGS. 4A and 4C, portions of the wall portions 105b along the longitudinal direction of the antenna 111 extend to the vicinity of the back surface of the bottom surface of the recessed portion 108b along a portion projecting toward the incident surface plate 101 in the recessed portion 108b (in the housing). As a result, the portions of the wall portions 105b along the longitudinal direction of the antenna 111 cover both side surfaces of the projecting portion.

As described above, the wall portions 105b extend to the vicinity of the back surface of the bottom surface of the recessed portion 108b so as to surround the side surfaces of the antenna 111 and have a structure like a closed space that encloses the antenna 111. In this structure, electromagnetic waves radiated from the antenna 111 are less likely to be released to the outside from the plate portion 105a and the wall portions 105b. That is, the antenna 111 except the opening through which the conductive line 115 connecting the antenna 111 and the wireless module 110 to each other passes is surrounded by the plate portion 105a and the wall portions 105b as well as the back housing portion 108 and the antenna window 113.

As a result, the portions not located in the direction of the antenna window 113 are substantially blocked electromagnetically. In the present embodiment, as illustrated in FIG. 4C, the components of electromagnetic waves released from the antenna 111 that are not directly oriented in the direction of the antenna window 113 are reflected on the inner surfaces of the wall portions 105b and the plate portion 105a pass through the antenna window 113. Accordingly, the electromagnetic waves from the antenna 111 can be efficiently released to the outside of the housing.

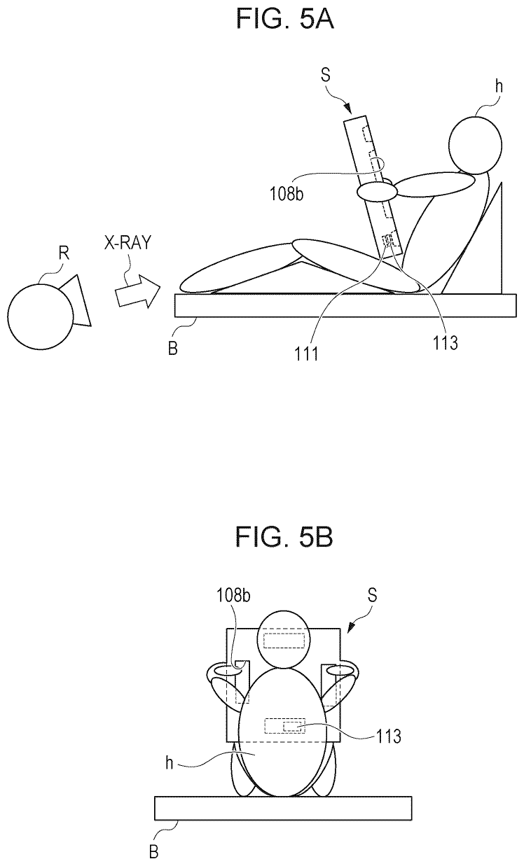

The following description will focus on X-ray radiography of, for example, a knee portion of the test subject by using the X-ray sensor according to the present embodiment. FIGS. 5A and 5B are schematic diagrams illustrating the X-ray radiography of the knee portion of the test subject by using the X-ray sensor according to the present embodiment. FIG. 5A is a side view in a radiography state, and FIG. 5B is a back view in the radiography state.

In this radiography method, X-rays released from an X-ray source R pass through the foot portion of the test subject h placed on a photographing table B in the axial direction, the X-rays are detected by the X-ray sensor S held by the test subject h, and X-ray radiography of the knee portion is performed. Here, the test subject h takes a picture while holding the X-ray sensor S with both hands. In the X-ray sensor S, the four recessed portions 108b extending along the four sides are formed in the back housing portion 108 and, in the disposition state illustrated in FIG. 5B, the antenna window 113 and the antenna 111 facing thereto in the housing are disposed in one of the recessed portions 108b that is disposed along the lower side. In the present embodiment, of the remaining recessed portions 108b, two recessed portions 108b extending along the left and right sides in the back housing portion 108 in the disposition state in FIG. 5B are used for the test subject h to put fingers when grasping the X-ray sensor S with both hands. The test subject h can stably hold the X-ray sensor S by putting fingers in the two recessed portions 108b extending along the left and right sides of the housing of the remaining recessed portions 108b. On the other hand, when the X-ray sensor is held as illustrated in FIG. 5A, the antenna 111 may come close to a high-risk portion of electromagnetic waves, such as an abdomen of the test subject h. Even in such a case, in the X-ray sensor S according to the present embodiment, since the antenna window 113 is disposed closer to the bottom surface of the recessed portion 108b and the distance between the test subject h and the antenna 111 increases, adverse effects of electromagnetic waves can be suppressed.

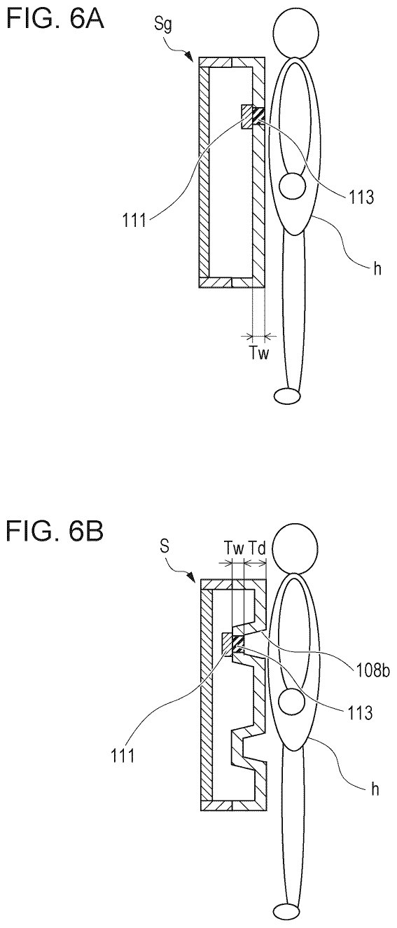

Reduction of adverse effects of electromagnetic waves on the test subject in the X-ray sensor according to the present embodiment will be described as compared with a comparative example. FIGS. 6A and 6B are schematic diagrams illustrating a case in which X-ray radiography of a chest portion is performed by using the X-ray sensor. FIG. 6A illustrates X-ray radiography by using an X-ray sensor according to the comparative example in which the recessed portion is not present in the back surface, and FIG. 6B illustrates radiography by using the X-ray sensor according to the present embodiment in which the recessed portion is present in the back surface.

In FIG. 6A, the distance relationship between the antenna 111, the antenna window 113, and the test subject h in an X-ray sensor Sg in the comparative example is illustrated. In general, the antenna window is often made of a resin, and a component made of a resin often has a thickness Tw of approximately 2 mm in terms of strength and moldability. Since the antenna is close to the antenna window, the distance from the antenna 111 to the test subject h can be close to approximately 2 mm.

In general, the thickness of an X-ray sensor is 16 mm or less. In the X-ray sensor S according to the present embodiment illustrated in FIG. 6B, the depth of the recessed portion 108b is preferably 4 mm or more to improve the placement conditions of fingers in the recessed portions along the left and right sides of the X-ray sensor S. Accordingly, when a depth Td of the recessed portion 108b is assumed to be 4 mm, which is the smallest value, if the thickness Tw of the antenna window 113 is 2 mm, the distance from the antenna 111 to the test subject h is approximately 6 mm.

The SAR indicates the effect of a magnetic field, and a magnetic field attenuates with distance when propagating in free space. The loss when distance from the antenna 111 to the test subject h is 2 mm in the typical X-ray sensor Sg is assumed to be L2. The loss when the distance from the antenna 111 to the test subject h is 6 mm in the X-ray sensor S according to the present embodiment is assumed to be L6. In this case, the difference Lo in loss between both sensors is calculated as follows.

Lo=L2−L6=20 log₁₀(2/6)=−20 log₁₀3≈−9.5(dB)

Since the difference Lo in loss is −9.5 dB, the SAR value of the X-ray sensor S according to the present embodiment can be suppressed to approximately 1/9 that of a general X-ray sensor. As described above, it can be seen that the SAR value is significantly reduced in the X-ray sensor S according to the present embodiment.

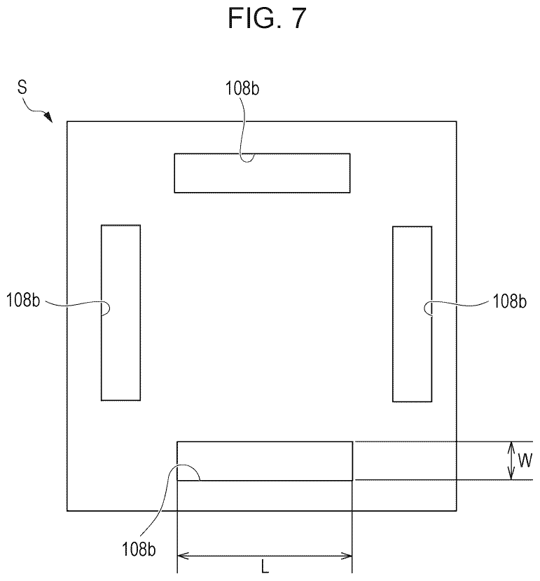

The proper size of the recessed portions formed in the back surface of the X-ray sensor according to the present embodiment will be described below. FIG. 7 is a diagram illustrating the back surface of the X-ray sensor according to the present embodiment. In FIG. 7, the width of the recessed portion in the lateral direction is assumed to be W, and the width in the longitudinal direction is assumed to be L.

First, the proper value of the width W of the recessed portion 108b in the lateral direction will be described. The thickness of the hand of an average person is approximately 17 mm, and when the thickness is 20 mm or more, fingers can be put in the recessed portion 108b. On the other hand, since the widths of the narrowest tips at the ends of the body, excluding the fingertips of hands and feet, are approximately 65 mm at the elbow and the heel, when the width is 40 mm or less, which is approximately twice the 20 mm described above, fingers can be put in the recessed portion 108b with sufficient margin. Accordingly, when the width W in the lateral direction is not less than 20 mm and not more than 40 mm, the recessed portion 108b is shaped to allow fingertips to be put thereinto to hold the X-ray sensor S and disallow high-risk portions, such as an abdomen and a head, that are susceptible to adverse effects of electromagnetic waves to be put thereinto.

Next, the value of the width L of the recessed portion 108b in the longitudinal direction will be described. When the X-ray sensor S is grasped, the recessed portion 108b may be partially covered with a hand. In this case, when the recessed portion 108b is completely covered with a hand, electromagnetic waves are less likely to be released to the outside and a communication failure is likely to occur. Here, since the width of a hand of an average person is approximately 80 mm, when the width L in the longitudinal direction is 80 mm or more, the recessed portion 108b is not be completely covered when the X-ray sensor S is grasped. Accordingly, when the width L in the longitudinal direction is 80 mm or more, electromagnetic waves can propagate through the gap between the recessed portion 108b and the test subject h, and accordingly, electromagnetic waves can be efficiently radiated to the outside of the X-ray sensor S.

As described above, the X-ray sensor S according to the present embodiment can sufficiently reduce the SAR value by reducing the risk of adverse effects on the human body caused by electromagnetic waves released from the antenna 111 without adding any special constituent members. Furthermore, electromagnetic waves can be efficiently released from the X-ray sensor S to the outside, and a good communication environment can be obtained. In addition, since the X-ray sensor S has a shape that can be easily grasped by the test subject, the X-ray sensor S is highly convenient.

### Third Embodiment

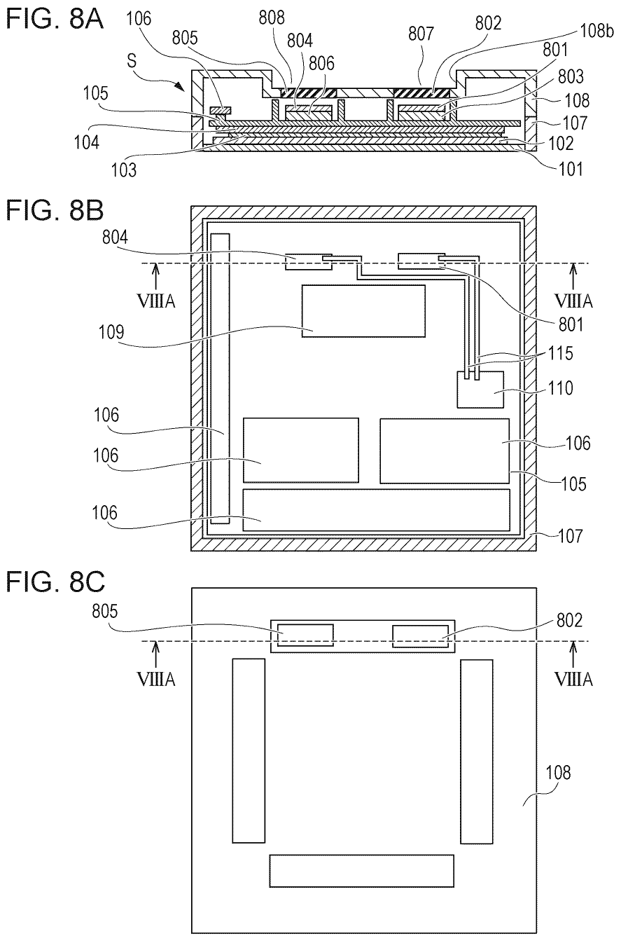

A third embodiment will be described with reference to FIGS. 8 to 10. It should be noted that components identical to those of the first and second embodiments will not be described.

FIGS. 8A, 8B, and 8C are schematic diagrams illustrating the X-ray radiography device (X-ray sensor) according to the present embodiment. FIG. 8A is a cross-sectional view taken along dotted line VIIIA-VIIIA in FIGS. 8B and 8C, FIG. 8B is a plan view illustrating the internal structure as viewed from the back surface of the X-ray incident surface, and FIG. 8C is a plan view as viewed from the back surface.

As illustrated in FIG. 8C, in the X-ray sensor S, the four recessed portions 108b extending along four sides are formed in the back housing portion 108 as in the second embodiment. In the present embodiment, in the bottom surface of one of the four recessed portions 108b that extends along the upper side of the back housing portion 108 in the state in FIG. 8C, a plurality of (for example, two) antenna windows, which are an antenna window A 802 and an antenna window B 805, is disposed side by side. As illustrated in FIG. 8A, the antenna window A 802 is disposed to block the opening A 807 formed in the bottom surface of the recessed portion 108b. The antenna window B 805 is disposed to block an opening B 808 formed in the bottom surface of the recessed portion 108b.

Here, as in the first and second embodiments, the antenna A 801 facing the antenna window A 802 is disposed and an antenna B 804 facing the antenna window B 805 is disposed in the housing. The antenna A 801 is placed on and fixed to an antenna base A 803 so as to be closer to the antenna window A 802, and the antenna B 804 is placed on and fixed to an antenna base B 806 so as to be closer to the antenna window B 805. Here, the antenna A 801 and antenna window A 802 are disposed at substantially the same position in plan view, and the antenna B 804 and the antenna window B 805 are disposed at substantially the same position in plan view.

In the present embodiment, the antenna A 801 or the antenna B 804 can be selectively used to communicate with the outside.

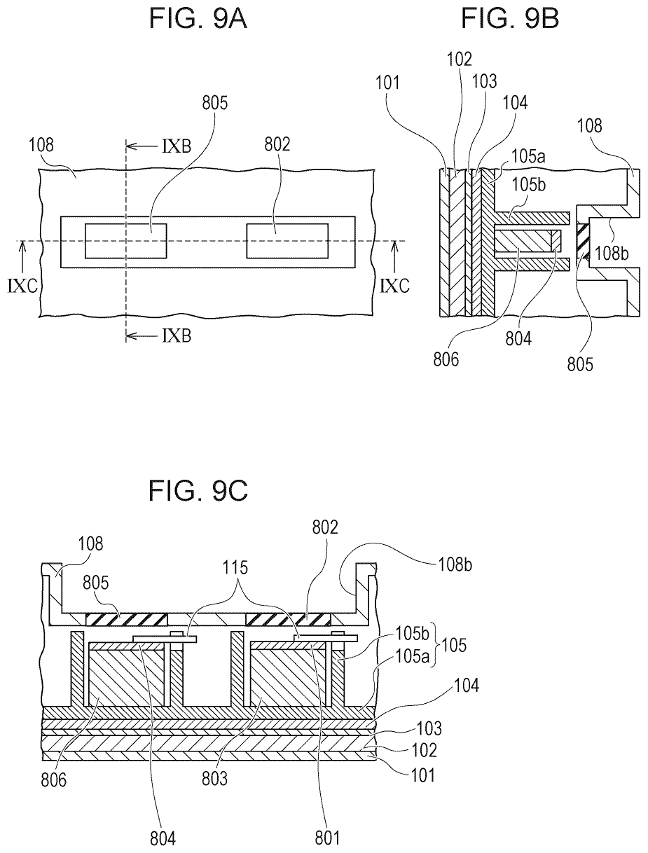

An aspect of the support base 105 in the present embodiment will be described below with reference to FIG. 9. FIGS. 9A, 9B, and 9C are schematic diagrams illustrating enlarged view of the vicinity of an antenna window in the present embodiment. FIG. 9A is a plan view, FIG. 9B is a cross-sectional view taken along dotted line IXB-IXB in FIG. 9A, and FIG. 9C is a cross-sectional view taken along dotted line IXC-IXC in FIG. 9A.

The support base 105 is mainly includes, as a basic constituent member, a plate portion 105a made of a material that shields electromagnetic waves, such as magnesium alloy, aluminum alloy, a steel material such as stainless steel, or a material such as CFRP, so that the sensor panel 104 and the like are not affected by electromagnetic waves radiated from the antenna B 804. In the present embodiment, the support base 105 includes the plate portion 105a and the wall portions 105b that are wall members formed integrally with the plate portion 105a and projecting toward the back housing portion 108 from the plate portion 105a. The wall portions 105b are shaped to surround the antenna base B 806 and the antenna B 804.

As illustrated in FIGS. 9A and 9B, portions of the wall portions 105b along the longitudinal direction of the antenna B 804 extend to the vicinity of the back surface of the bottom surface of the recessed portion 108b along a portion projecting toward the incident surface plate 101 in the recessed portion 108b (in the housing). As a result, the portions of the wall portions 105b along the longitudinal direction of the antenna B 804 cover both side surfaces of the projecting portion. As illustrated in FIGS. 9A and 9C, portions of the wall portions 105b along the lateral direction of the antenna B 804 extend to the vicinity of the back surface of the bottom surface of the recessed portion 108b below the bottom surface and cover both side surfaces in the lateral direction of the antenna B 804. Here, portions of the wall portions 105b have openings through which the conductive lines 115 connecting the antenna B 804 and the wireless module 110 to each other passes.

As described above, the wall portions 105b extend to the vicinity of the back surface of the bottom surface of the recessed portion 108b so as to surround the side surfaces of the antenna B 804 and have a structure like a closed space that encloses the antenna B 804. As a result, electromagnetic waves radiated from the antenna B 804 are less likely to be released to the outside from the plate portion 105a and the wall portions 105b. That is, the antenna B 804 except the openings through which the conductive lines 115 connecting the antenna B 804 and the wireless module 110 to each other pass is surrounded by the plate portion 105a and the wall portions 105b as well as the back housing portion 108 and the antenna window B 805. As a result, the portions not located in the direction of the antenna window B 805 are substantially blocked electromagnetically.

In the present embodiment, the components of electromagnetic waves released from the antenna B 804 that are not directly oriented in the direction of the antenna window B 805 are reflected on the inner surfaces of the plate portion 105a and the wall portions 105b that substantially blocks the antenna B 804 and pass through the antenna window B 805. Accordingly, the electromagnetic waves from the antenna B 804 can be efficiently released to the outside of the housing.

The relationship of the antenna B 804, the antenna window B 805, and the antenna base B 806 described above is also applicable to the antenna A 801, the antenna window A 802, and the antenna base A 803. Also in this case, the electromagnetic waves from the antenna A 801 can be efficiently released to the outside of the housing.

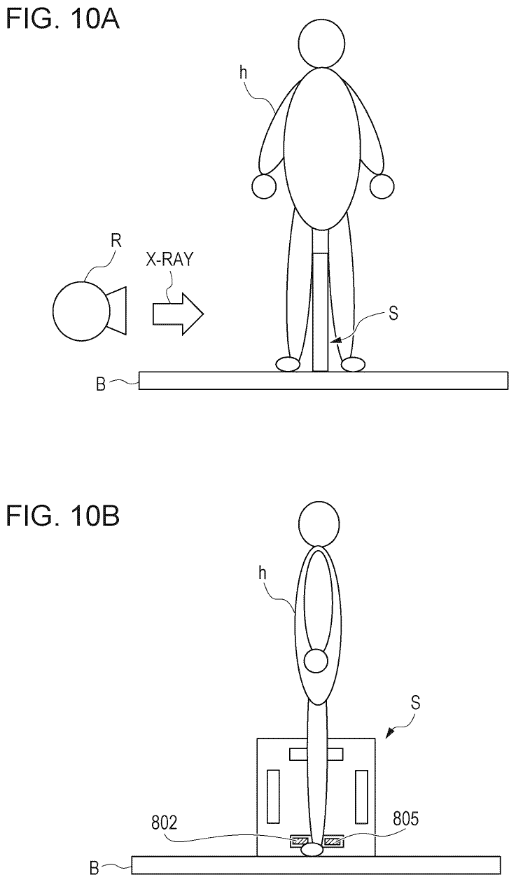

The following description will focus on X-ray radiography of, for example, a foot portion of the test subject by using the X-ray sensor according to the present embodiment. FIGS. 10A and 10B are schematic diagrams illustrating X-ray photography of a foot portion of the test subject. FIG. 10A is a front view illustrating a radiography state and FIG. 10B is a side view illustrating a back side of the X-ray sensor in the radiography state.

In this radiography method, X-rays released from the X-ray source R pass through the foot portion of the test subject h standing on the photographing table B from the side surface, and the X-rays are detected by the X-ray sensor S held between both feet of the test subject to perform X-ray radiography of the foot portion. Since the X-ray sensor S is held between both feet of the test subject h, when only one antenna window is provided in the bottom surface of the recessed portion 108b, a portion of the antenna window is blocked by the foot portion of the test subject, thereby causing a problem in communication with the antenna. In the present embodiment, as illustrated in FIG. 10B, the antenna window A 802 and the antenna window B 805 are disposed side by side on the bottom surface of one recessed portion 108b. In this structure, both antenna windows are prevented from being blocked by a portion of the body of the test subject h at the same time, and good communication is enabled by selectively using an antenna with a better communication state among the antenna A 801 and the antenna B 804.

As a result, the X-ray sensor S according to the present embodiment can sufficiently reduce the SAR value by reducing the risk of adverse effects on the human body caused by electromagnetic waves released from the antenna 111 without adding any special constituent members. In addition, electromagnetic waves can be efficiently released from the X-ray sensor S to the outside, and a good communication environment can be obtained. Furthermore, the risk of reducing communication performance due to the partial blockage of recessed portion 108b during use of the X-ray sensor S can be suppressed.

In addition, the X-ray sensor S has a shape that can be easily grasped by the test subject, the X-ray sensor S is highly convenient.

### Fourth Embodiment

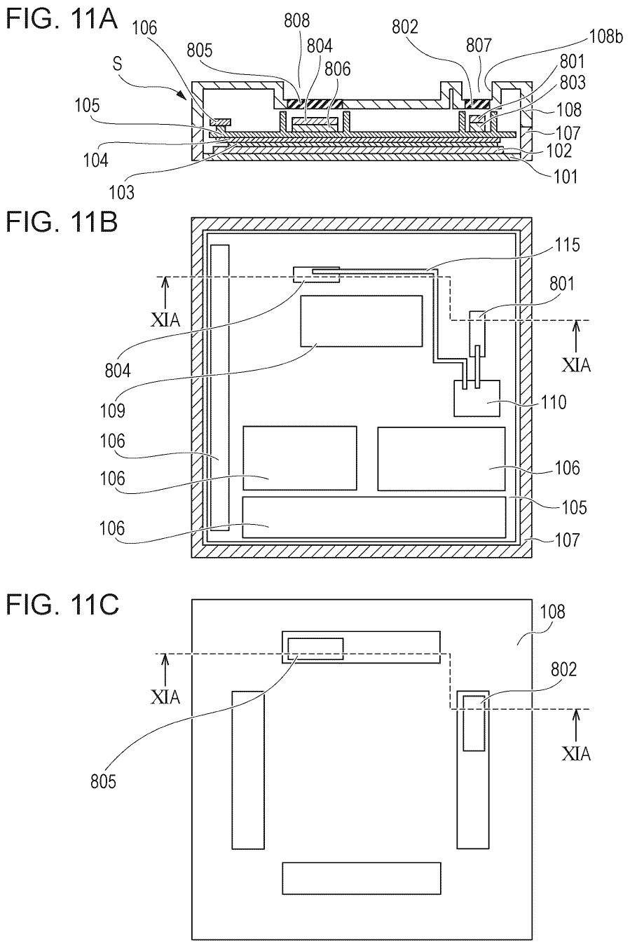

A fourth embodiment will be described with reference to FIG. 11. It should be noted that components identical to those of the first to third embodiments will not be described.

FIGS. 11A, 11B, and 11C are schematic diagrams illustrating the X-ray photography device (X-ray sensor) according to the present embodiment. FIG. 11A is a cross-sectional view taken along dotted line XIA-XIA in FIGS. 11B and 11C, FIG. 11B is a plan view illustrating an internal structure as viewed from the back surface of the X-ray incident surface, and FIG. 11C is a plan view as viewed from the back surface.

As illustrated in FIG. 11C, the X-ray sensor S has the four recessed portions 108b extending along the four sides in the back housing portion 108 as in the second and third embodiments. In the present embodiment, two of the four recessed portions 108b each have an antenna window. Specifically, in the state in FIG. 11C, one antenna window A 802 is disposed in the bottom surface of the recessed portion 108b extending along the right side of the back housing portion 108. In addition, in the state in FIG. 11C, one antenna window B 805 is disposed in the bottom surface of the recessed portion 108b extending along the upper side of the back housing portion 108. As illustrated in FIG. 11A, the antenna window A 802 and the antenna window B 805 are disposed to block the openings A 807 and B 808 formed in the bottom surface of the recessed portion 108b. Here, as in the third embodiment, in the housing, the antenna A 801 facing the antenna window A 802 is disposed, and the antenna B 804 facing the antenna window B 805 is disposed. The antenna A 801 is placed on and fixed to the antenna base A 803 so as to be closer to the antenna window A 802, and the antenna B 804 is placed on and fixed to the antenna base B 806 so as to be closer to the antenna window B 805. Here, the antenna A 801 and the antenna window A 802 are disposed at substantially the same position in plan view, and the antenna B 804 and the antenna window B 805 are disposed at substantially the same position in plan view.

In the present embodiment, the antenna A 801 and the antenna B 804 are used to perform so-called MIMO (multi-input multi-output) communication that simultaneously transmits different types of information. MIMO communication is a communication method that increases the amount of communication by simultaneously transmitting different types of information by using a plurality of antennas. MIMO communication has a benefit in that a plurality of antennas can simultaneously communicate different types of information but has a risk in that the electromagnetic waves radiated from one antenna have adverse effects on another antenna and causes reduction in communication performance.

In the present embodiment, since the antenna A 801 and the antenna B 804 are located in different recessed portions 108b, they are relatively separated from each other and do not significantly affect each other, thereby suppressing the degradation of communication performance.

In addition, in the present embodiment, the support base 105 includes the wall portion 105b as in the third embodiment. The wall portions 105b extend to the vicinity of the back surface of the bottom surface of each of the recessed portions 108b so as to surround the side surfaces of the antenna A 801 and the antenna B 804 and have a structure like a closed space that encloses the antenna A 801 and the antenna B 804. As a result, the antenna A 801 and the antenna B 804 are electromagnetically shielded from each other by the wall portions 105b. That is, the antenna A 801 except the openings through which the conductive lines 115 connecting the antenna A 801 and the wireless module 110 to each other pass is surrounded by the plate portion 105a and the wall portions 105b as well as the back housing portion 108 and the antenna window A 802. The antenna B 804 except the openings through which the conductive lines 115 connecting the antenna B 804 and the wireless module 110 to each other pass is surrounded by the plate portion 105a and the wall portions 105b as well as the back housing portion 108 and the antenna window B 805.

As a result, the portions not located in the direction of the antenna window A 802 are substantially blocked electromagnetically. The portions of the antenna B 804 not located in the direction of the antenna window B 805 are substantially blocked electromagnetically. Accordingly, the electromagnetic waves released from the antenna A 801, propagating through the X-ray sensor S, and reaching the antenna B 804, and the electromagnetic waves released from the antenna B 804, propagating through the X-ray sensor S, and reaching the antenna A 801 are both low. Accordingly, the risk of hindering MIMO communication is sufficiently reduced.

As described above, the X-ray sensor S according to the present embodiment can sufficiently reduce the SAR value by reducing the risk of adverse effects on the human body caused by electromagnetic waves released from the antenna 111 without adding any special constituent members. In addition, electromagnetic waves can be efficiently released from the X-ray sensor S to the outside, and a good communication environment can be obtained. Furthermore, during high-speed communication using MIMO communication, adverse effects of electromagnetic waves from the antenna A 801 and the antenna B 804 on communication through these antennas are also reduced.

The first to third embodiments have been described above, but these embodiments can be combined with each other as appropriate.

For example, in the first embodiment, the support base 105 may include the plate portion 105a and the wall portions 105b, and the wall portions 105b may surround the side surface of the antenna 111 as in the second embodiment. In this structure, the X-ray sensor S according to the first embodiment can also radiate, to the outside of the X-ray sensor S, the electromagnetic waves released from the antenna 111.

In addition, for example, the third embodiment can be combined with the fourth embodiment. In this case, the X-ray sensor S includes a plurality of antennas, some of the plurality of antennas are disposed to correspond to one recessed portion 108b, and the remaining antennas are disposed to correspond to another recessed portion 108b.

Specifically, the antenna window A 802 and the antenna window B 805 are disposed side by side in one recessed portion 108b along, for example, the upper or lower side of the four recessed portions 108b extending along the four sides in the back housing portion 108. The antenna A 801 facing the antenna window A 802 in the housing is disposed in the antenna window A 802, and an antenna B 804 facing the antenna window B 805 in the housing is disposed in the antenna window B 805.

In addition, the antenna window A 802 and the antenna window B 805 are disposed side by side in one recessed portion 108b along, for example, the right or left side of the four recessed portions 108b. The antenna A 801 facing the antenna window A 802 in the housing is disposed in the antenna window A 802, and an antenna B 804 facing the antenna window B 805 in the housing is disposed in the antenna window B 805.

Here, the two antenna windows A 802 and B 805 disposed in one recessed portion 108b along the upper or lower side can be selectively used for communication with the outside by using either of them. Similarly, the two antenna windows A 802 and B 805 disposed in one recessed portion 108b disposed along the right side or the left side can be selectively used to communicate with the outside by using one of these antenna windows. In addition, in the antenna window A 802 and the antenna window B 805 disposed in the recessed portion 108b along the upper or lower side and the antenna window A 802 and the antenna window B 805 disposed in the recessed portion 108b along the right or the left side of the recessed portion 108b, MIMO communication that simultaneously communicates different types of information is performed. In the structure described above, the risk of reducing communication performance due to the partial blockage of the recessed portion 108b during use of the X-ray sensor S is suppressed. At the same time, adverse effects on communication caused by electromagnetic waves released from the four antennas in high-speed communication through MIMO communication are also reduced.

The embodiments described above are only examples of implementation of the present disclosure and should not be considered as restrictions on the technical scope of the present disclosure. That is, the present disclosure can be implemented in various forms without departing from the technical concept or the main features.

### OTHER EMBODIMENTS

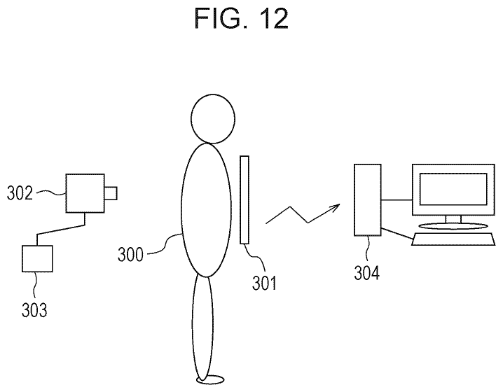

The radiation detecting devices (X-ray sensors S) according to the embodiments described above are applicable to a radiography system as illustrated in, for example, FIG. 12.

This radiography system includes a radiography device 301 that is one of the X-ray sensors S according to one of the embodiments described above, a radiation generating device 302, a control unit 303, and a control and calculation processing unit 304. The control unit 303 controls the output of radiation (X-rays here) from the radiation generating device 302. The radiography device 301 communicates wirelessly with the control and computation processing unit 303. In this radiography system, radiation is output to the test subject 300, which is a photographic subject, from the radiation generating device 302. The radiography device 301 detects the radiation having been transmitted through the test subject 300 and generates image information. Information, such as image information, generated by the radiography device 301, is transmitted as a wireless signal from the antenna of the radiography device 301 and received by the control and computation processing unit 304. The control and computation processing unit 304 performs desired computation processing in accordance with the received information and performs diagnosis.

This radiography system can perform more accurate diagnosis by using the radiography device 301 that can sufficiently reduce the SAR value by reducing the adverse effects of communication electromagnetic waves on the test subject and the like without need for additional constituent members as SAR measures.

According to the present disclosure, it is possible to perform miniaturization and weight reduction of the device without need for additional constituent members as SAR measures and sufficiently reduce the SAR value by reducing adverse effects of communication electromagnetic waves on the test subject and the like.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2023-174657, filed Oct. 6, 2023, which is hereby incorporated by reference herein in its entirety.

What is claimed is:

1. A radiography device comprising:
a sensor panel configured to generate image information from incident radiation;
an antenna configured to wirelessly transmit the image information generated by the sensor panel; and
a housing configured to house the sensor panel and the antenna,
wherein the housing has a recessed portion recessed toward an inside, the recessed portion having a transmission portion configured to transmit an electromagnetic wave, and
the antenna is disposed to face the transmission portion.

2. The radiography device according to claim 1,
wherein the housing has an incident surface on which radiation is incident and a back surface facing away from the incident surface, and
the recessed portion is formed in the back surface.

3. The radiography device according to claim 1,
wherein the transmission portion is a window portion that blocks an opening formed in the recessed portion with a material through which an electromagnetic wave can be transmitted.

4. The radiography device according to claim 1, further comprising:
a display label configured to be fitted to the recessed portion, the display label being made of a material through which an electromagnetic wave can be transmitted.

5. The radiography device according to claim 1, further comprising:
a placement portion on which the antenna is placed.

6. The radiography device according to claim 1, further comprising:
a support plate configured to support the antenna, the support plate being made of a material that shields an electromagnetic wave.

7. The radiography device according to claim 6,
wherein the support plate includes a wall portion that projects toward a surface of the housing in which the recessed portion is formed.

8. The radiography device according to claim 7,
wherein the wall portion surrounds the antenna.

9. The radiography device according to claim 8,
wherein a portion of the wall portion extends along a side surface of the recessed portion.

10. The radiography device according to claim 2,
wherein the recessed portion is disposed in a center portion of the back surface.

11. The radiography device according to claim 2,
wherein the recessed portion extends along a portion of an outer edge of the back surface of the housing.

12. The radiography device according to claim 11,
wherein four recessed portions along four sides constituting the outer edge are formed, the recessed portion being one of the four recessed portions.

13. The radiography device according to claim 12,
wherein a width of the recessed portions in a lateral direction is 40 mm or less.

14. The radiography device according to claim 12,
wherein a width of the recessed portions in a longitudinal direction is 80 mm or more in a longitudinal direction.

15. The radiography device according to claim 1,
wherein the antenna is one of at least two antennas, and
the at least two antennas are disposed to correspond to the recessed portion.

16. The radiography device according to claim 15,
wherein one of the at least two antennas is selected to enable communication.

17. The radiography device according to claim 1,
wherein the antenna is one of at least two antennas, and
at least one antenna of the at least two antennas is disposed to correspond to the recessed portion, and a remaining antenna of the at least two antennas is disposed to correspond to another recessed portion.

18. The radiography device according to claim 17,
wherein information transmitted by the at least one antenna differs from information transmitted by the remaining antenna.

19. A radiography system comprising:
a radiation generating device configured to radiate radiation to a subject;
the radiography device according to claim 1; and
an arithmetic processing device configured to wirelessly communicate with the antenna of the radiography device and performs predetermined arithmetic processing in accordance with image information generated by the radiography device.
